(19) United States
(12) Patent Application Publication
(10) Pub. No.: US 2018/0021124 A1
(43) Pub. Date: Jan. 25, 2018

(54) BODY PART SUPPORT DEVICE AND METHOD

(71) Applicant: Exsomed International IP, LLC, Avarua (CK)

(72) Inventors: Lloyd P. Champagne, Phoenix, AZ (US); Jozef Zoldos, Phoenix, AZ (US)

(21) Appl. No.: 15/214,412

(22) Filed: Jul. 19, 2016

(51) Int. Cl. A61F 2/08; A61B 17/17; A61B 17/16

(52) U.S. Cl. CPC A61F 2/0811; A61B 17/1686; A61B 17/1714; A61F 2002/0882; A61F 2002/0852; A61B 2017/1782

(57) ABSTRACT

A device and method for supporting a body part by another body part. The device includes a first (or proximal) button, a second (or distal) button, and a suture connecting the first button to the second button, with there being a space between the first button and second button. An opening is formed through two body parts, and the second button and suture are moved through the opening until the second button is outside of the opening and is seated against the outer (distal) side of the second body part. The first button is then seated against the outer (proximal) side of the first body part, so that one of the body parts provides support for the other. The first button may be attached to the suture after the second button is seated on the distal side of the second body part.

### FIELD OF THE INVENTION

The present invention relates to the field of supporting one body part, especially a bone, by a connection to another body part, especially a bone.

### BACKGROUND

CMC arthritis or thumb base carpometacarpal arthritis is usually a degenerative process that affects the base of the thumb as one ages. Usually, the symptom is progressive pain when the thumb is moved. Diagnosis is based on clinical findings of pain at the thumb base with movement, and radiographic findings of arthritis. The typical patient is female and in her forties.

Conservative treatments include anti-inflammatories, splinting, and cortisone injections. At some point, if the pain is severe and unrelieved with conservative treatment, surgery is needed.

Surgical options for treatment of CMC joint instability and arthritis include trapezium excision, CMC fusion, and Ligament Reconstruction and Tendon Interposition (LRTI). One surgical technique for CMC arthritis is joint resection of the CMC joint with suspension of the thumb metacarpal to the index metacarpal. Joint resection is performed by excising the degenerative trapezium bone which sits below the metacarpal. The degenerative articular cartilage at the CMC position is removed with trapeziectomy. The degenerative cartilage on the metacarpal base may or not need to be removed. When the trapezium is removed, the floor or foundation of the thumb is also removed and a gap is created at the site of the previous trapezium. This now unstable thumb is suspended from the index finger base by creating a suspension using tendon from the patient's forearm. Extra tendon is usually balled up and placed into the trapezium defect to provide space fill and padding.

U.S. Pat. No. 8,961,575, and U.S. Patent Application Publication No. 2011/0224729, describe devices and methods for repairing a CMC joint.

### SUMMARY

The present invention includes devices and methods to support a body part, such as the metacarpal bone of the thumb, with another body part, such as the metacarpal bone of the index finger. In one particular application, embodiments of the invention may be used to treat CMC arthritis and/or CMC thumb instability. In a method according to the invention, a tunnel (or opening) is first formed through (a) a body part to be supported, and (b) a body part that provides the support. The present methods and devices are intended to replace the suspension portion of suspension arthroplasty. By employing a device of the invention to support the thumb metacarpal bone by the index metacarpal bone, the need to harvest a tendon from the patient is negated. This should result in less patient morbidity, less patient pain, less operative time, and potential cost savings based on shorter operative time.

The opening is preferably formed by using a K-wire and then placing a cannulated drill bit over the K-wire to enlarge the opening formed by the K-wire. In an application wherein one bone is supported by another, holes are preferably formed in adjoining bones (such as two adjoining metacarpals; for example, the first and second metacarpals).

A device according to the invention includes at least a first (or proximal) button, a second (or distal) button, and a suture connected initially to at least the second button. There is a space between the first button and the second button. The second button preferably has a first position and a second position, and may be biased towards the second position. In the repair of a CMC joint, the second button and suture are pushed through the opening until the second button is outside of the second metacarpal. The second button then moves to its second position so that it cannot pass back through the opening, but is seated against the outside of the second metacarpal, which is the distal side of the second metacarpal bone in relation to the formation of the opening. The second button may move by itself to the second position if it is biased towards the second position, or be moved to the second position by a surgeon, such as by pulling on the suture. The first button is then seated against the outside (which is the proximal side in relation to the formation of the opening) of the first metacarpal bone. If the first button is not initially connected to the suture, it is connected to the suture at this time. If the first button was initially connected to the suture, it is tightened so as to be seated against the first metacarpal. This may be done by loosening the suture attached to the first button, repositioning the first button, and then tightening the suture to seat the first button against the first metacarpal bone.

Thus, one method according to aspects of the invention for supporting a body part, such as a bone, includes the steps of: (i) providing a suture having a first end attached (optionally) to a first button, and a second end attached to a second button; (ii) passing the suture and second button through an opening (such as an opening through the first metacarpal and second metacarpal) in a body part to be supported and a body part that provides the support; (iii) moving the second button outside of the opening and outside of the distal body portion, such as outside of the second metacarpal; (iv) moving the second button to its second position (which may occur automatically or by manipulation by a surgeon), whereby the second button is seated against the outside surface of the distal body portion and does not move back through the opening; (v) optionally, adjusting the space between the two body parts; and (vii) securing the first button in place outside of the proximal body portion, so that it does not move through the opening.

### BRIEF DESCRIPTION OF THE DRAWINGS

FIG. 1 illustrates a hand showing the CMC joint.

FIG. 2 illustrates a close-up view of the CMC joint illustrating arthritic changes.

FIG. 3 illustrates a tendon repair of a CMC joint.

FIG. 3A illustrates prior art CMC suspension arthroplasty.

FIG. 3B illustrates an incision formed in the hand to begin a method according to aspects of the invention.

FIG. 3C illustrates a perspective view of a button that may be used in aspects of preferred embodiments of the invention.

FIG. 3D illustrates an embodiment of a device according to aspects of the present invention.

FIG. 4 illustrates an inserter tube according to aspects of the present invention.

FIG. 5 illustrates a suture with a second button according to aspects of the present invention.

FIG. 6 illustrates a K-wire.

FIG. 7 is a close-up view of the suture and second button of FIG. 5.

FIG. 8 illustrates the travel of a K-wire through the first and second metacarpal bones.

FIG. 9 illustrates the travel of a cannulated drill bit over the K-wire of FIG. 8.

FIG. 10 illustrates removal of the K-wire leaving behind the cannulated drill bit on the opening.

FIG. 11 illustrates moving a device according to aspects of the invention through the opening via the cannulated drill bit.

FIG. 12 illustrates attaching the first button to the suture outside of the opening.

FIGS. 13-14 illustrates a device according to aspects of the invention with the first button attached prior to pushing the second button and suture through the opening, and showing a cannulated drill bit having a longitudinal slit.

FIGS. 15 and 15A illustrates another button that may be used in embodiments of the invention.

### DETAILED DESCRIPTION OF PREFERRED EMBODIMENTS

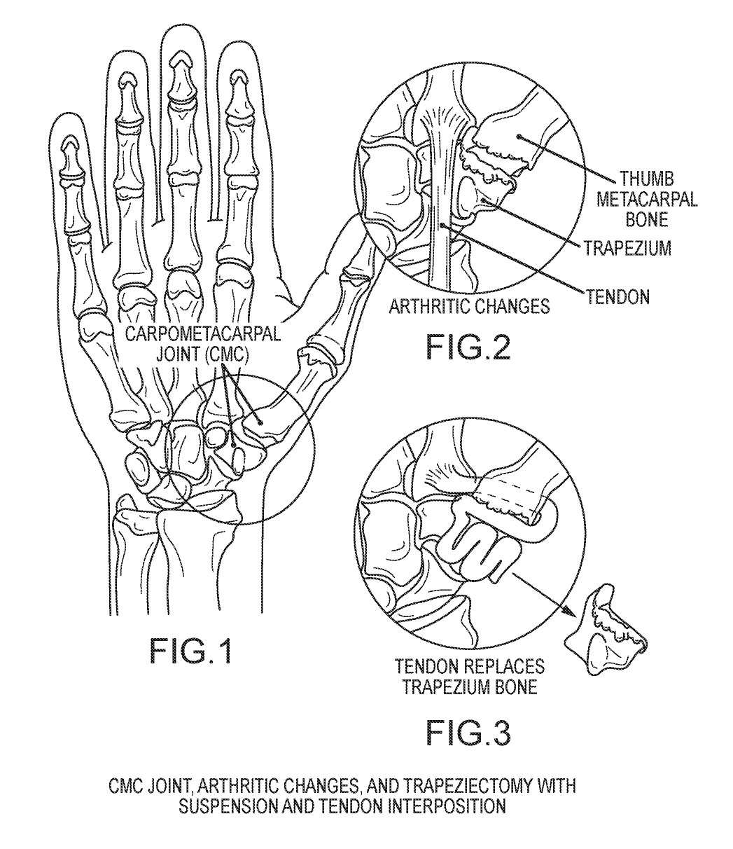

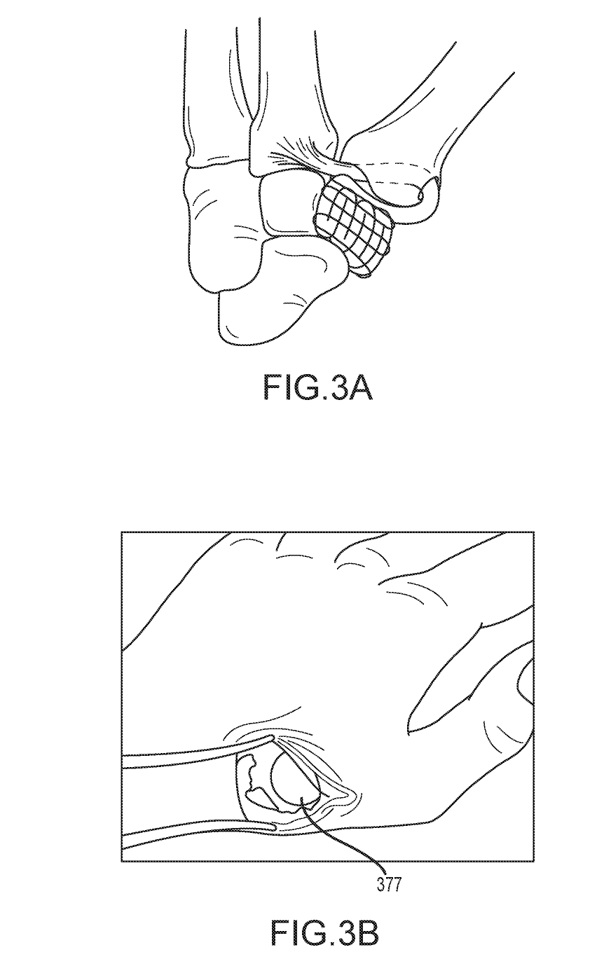

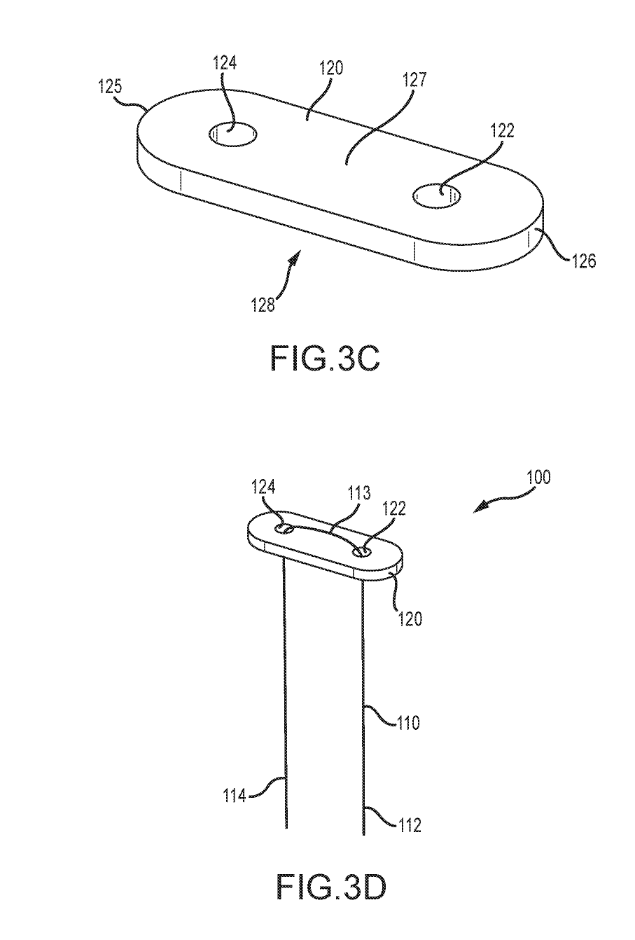

Referring now to the drawings, wherein the purpose is to describe preferred embodiments of the invention and not to limit its scope, FIGS. 1-3 illustrate, respectively, a healthy CMC joint, an arthritic CMC joint, and a CMC joint repair using a tendon, which is usually harvested from the arm. FIG. 3A illustrates a CMC suspension arthroplasty. FIG. 3B shows a typical incision in a hand made prior to supporting a thumb (first) metacarpal bone in accordance with aspects of the invention.

The present inventions can be used, among other things, to replace or augment tendon repair techniques in corrective surgery for treatment of CMC arthritis, thumb CMC instability and revisions with proximal migration after failed tendon reconstruction. In an exemplary embodiment, device 100 of the present invention is employed in metacarpal repairs and instabilities, for example, in stabilizing the thumb metacarpal (first metacarpal) following removal or partial resection of the trapezium, and/or for treatment of carpometacarpal arthrosis and instability (for example, between two metacarpals, such as the first and second metacarpals).

In certain applications, trapezia resection or removal may be conducted prior to placement of device 100 in the repair of a CMC joint.

In the embodiment shown, device 100 supports the thumb (first) metacarpal by the index finger (second) metacarpal. Device 100, however, can be modified in size, length and strength to support other bones or body parts, by other bones or body parts. For example, a device and method according to the insertion may be used to support bones in the foot or lower leg by other bones in the foot or lower leg. Or, to support a tendon by a bone.

A device and method according to the invention uses at least two buttons—a first button and a second button. As used herein, “button” refers to any structure that can be seated against and transfer a load necessary to support a body part without damaging the body part to which it is seated.

FIG. 3C illustrates a button 120 that may be used for either the first button, second button or both, of device 100. Button 120 as shown is an oblong shaped plate with chamfered or rounded corners and edges. Button 120 has a length that extends from proximal end 125 to distal end 126. Button 120 has a width that is shorter than its length and thickness, a front face 127 and back face 128, wherein the distance between the front face 127 and back face 128 is less than the width of button 120. Alternatively, the distance between front face 12 and back face 127, 128 is greater than the width of button 120. Button 120 is of any suitable size, shape and material to be used to support one body part by another body part, and its size and configuration may be modified depending on the body part on which it is ultimately seated.

Button 120 preferably includes at least one hole, and as shown has two holes 122 and 124 that extend from front face 127 to back face 128. Hole 122 is located near distal end 126. Hole 124 is located near proximal end 125. The one or more holes may, however, be at any suitable location on button 120. Further, a different structure may be used to attach the suture (described below) to button 120. Holes 122 and 124 are of sufficient size to allow suture 110 to pass through.

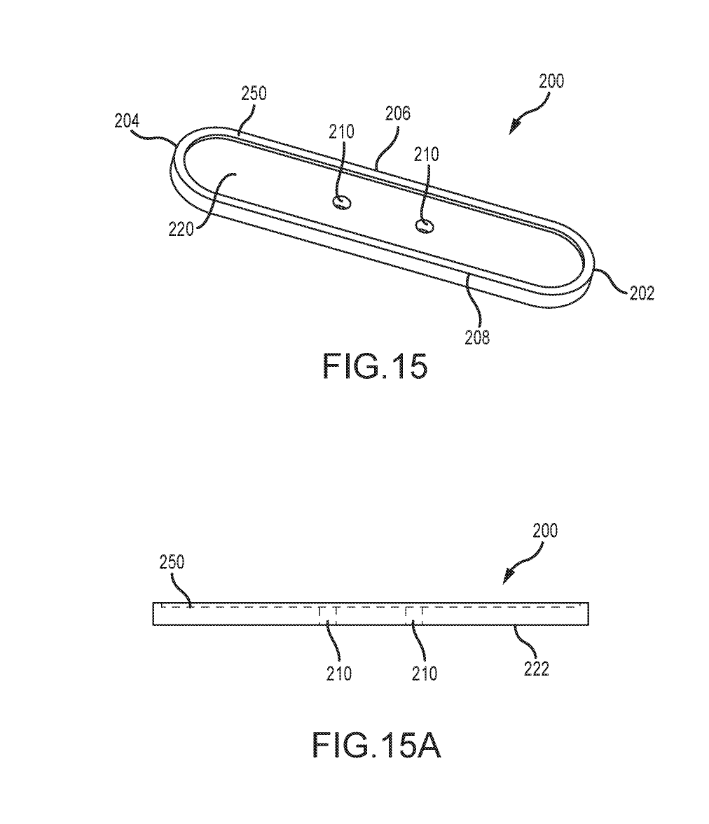

One alternative button 200, shown in FIGS. 15 and 15A, is oblong and rod-shaped. It preferably has two holes 210 that as shown are near the center of button 200. Button 200 may have a raised outer ridge 250, and has a first surface 220, an opposite surface 222, a first end 202, a second end 204, a first side 206, and a second side 208. Device 100 may include a first button 120 and second button 200, a first button 120 and second button 120, a first button 200 and a second button 120, or a first button 200 and second button 200.

FIG. 3D illustrates an exemplary embodiment of device 100 that includes a suture 110 and a second button, which as shown is a button 120. Suture 110 is a threadlike material that is commonly employed in surgery to hold tissue or bone together. The suture material is preferably flexible and can stretch when force is applied to it. Different types of sutures may be used to practice the invention depending upon the procedure and body parts involved.

As shown in FIG. 3D, in this embodiment of device 100, suture 110 passes through first hole 122 of button 120 and through second hole 124. As a result, suture 110 includes a first leg 112 and a second leg 114. Suture 110 also includes connecting section 113 that extends between first hole 122 and second hole 124 along front face 127 of button 120 in the embodiment shown.

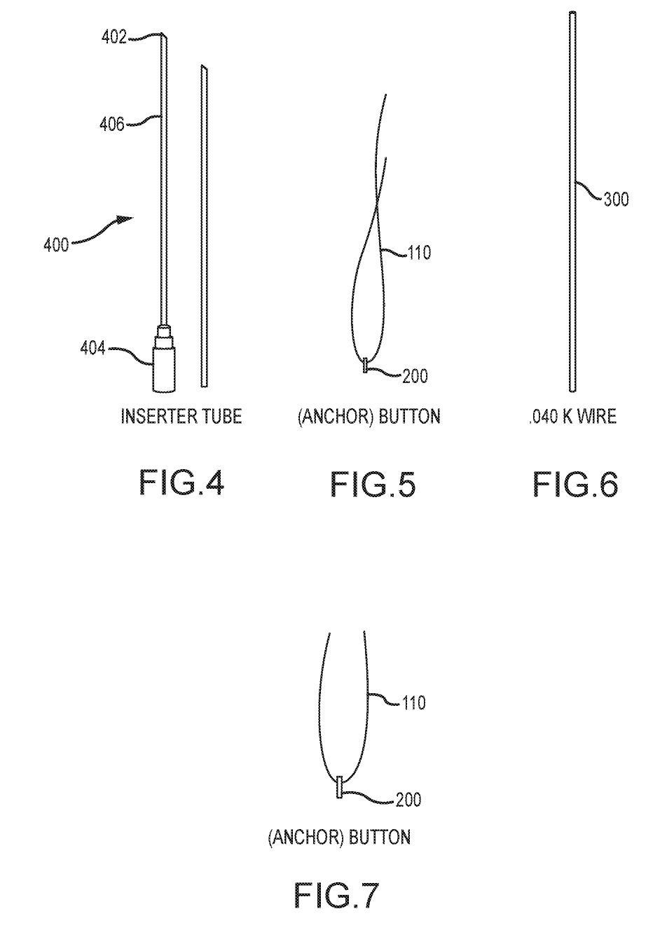

FIGS. 5 and 7 show other views of the device 100, but with the second button being button 200. FIG. 6 shows a K-wire 300 having a diameter of 0.040″, which is a device known by those skilled in the art. FIG. 4 shows an inserter tube 400 that has a first (or distal) end 402, a second (or proximal) end 404, and an elongated body 406.

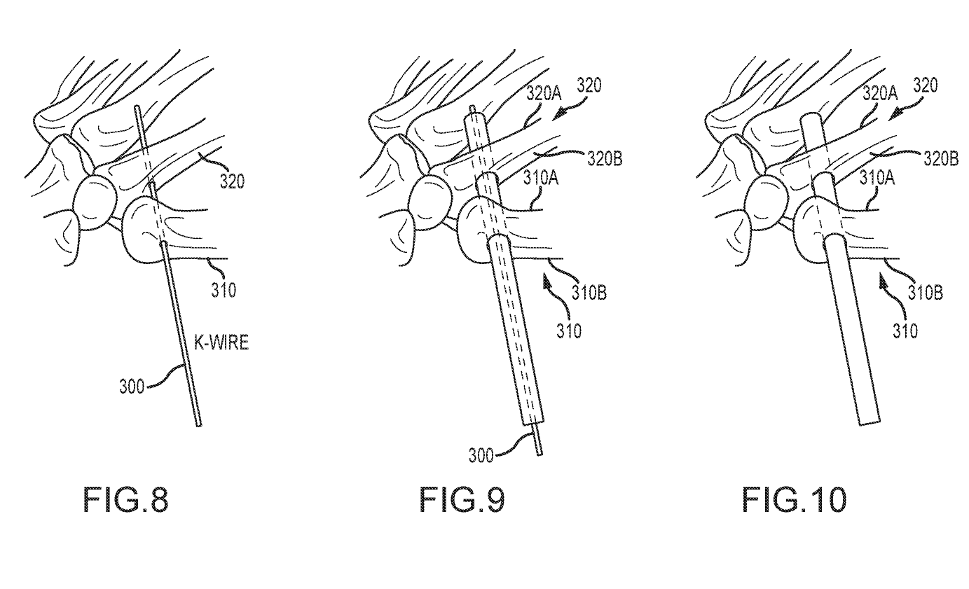

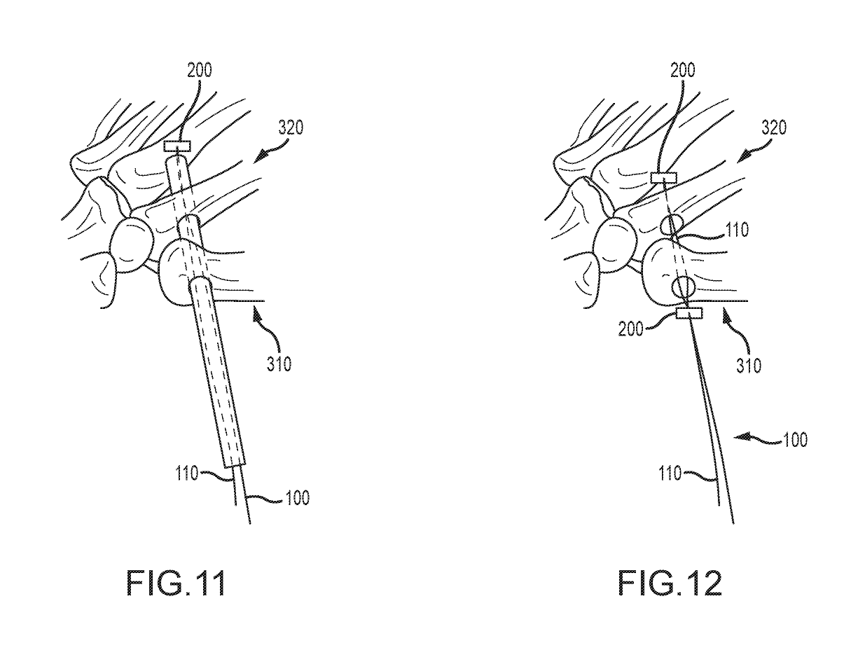

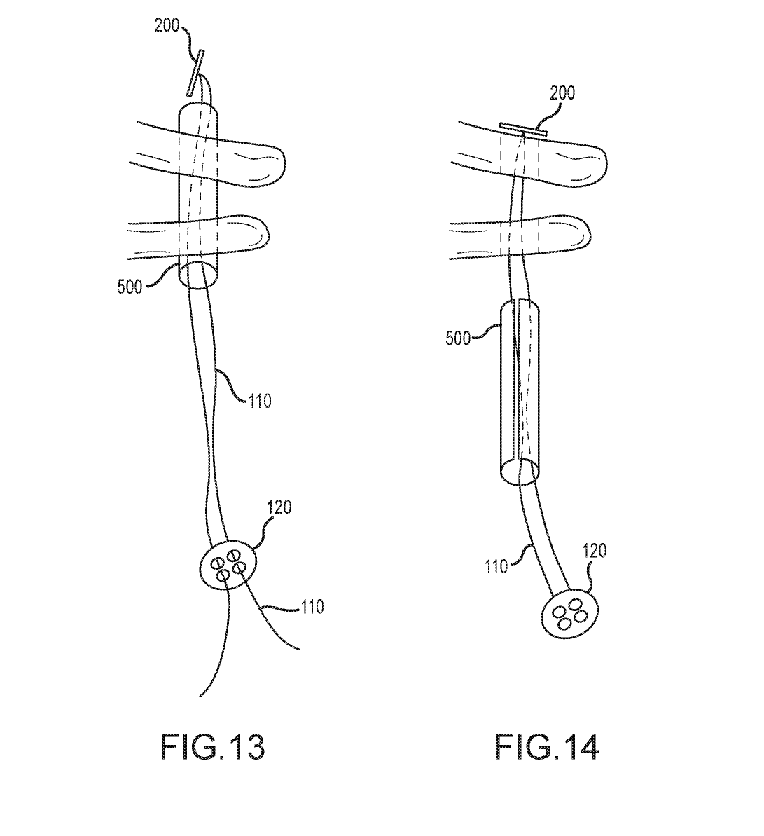

FIGS. 8-12 illustrate an exemplary CMC repair using device 100 or 150 (which has the first button connected to the suture prior to the second button being seated, and is shown in FIGS. 13 and 14) of the present invention. In one exemplary embodiment, device 100 or device 150 is employed to connect two metacarpals, for example, first and second metacarpals 310, 320. An opening is formed using K-wire 300 to push through first metacarpal 310 and second metacarpal 320. It is preferred that a cannulated drill bit is then positioned over K-wire 300 and drilled through the first metacarpal 310 and the second metacarpal 320, as shown in FIGS. 8 and 9. In this preferred embodiment, as shown in FIG. 10, the K-wire 300 is then removed and the cannulated drill bit remains in the opening. Alternatively, inserter tube 400 is positioned in the opening with end 404 at the proximal side 310B of first metacarpal 310, and end 402 at the distal side 320A of second metacarpal 320.

Device 100 or device 150 is then positioned into the opening (which means the opening itself, or the cannulated drill bit in the opening, or the inserter tube 400 in the opening) and pushed through using the K-wire 300 (or another K-wire). The second button (120 or 200) is in its first position, wherein it is generally axially aligned with the longitudinal axis of the opening so that it can pass through the opening. When the second button completely exits the opening and is outside of the second distal side 320A, it moves to its second position. It the second position, the second button is generally aligned cross axially with the longitudinal axis of the opening. The second button may be biased towards the second position and automatically move to the second position once outside of the opening. Or, surgeon may move the second button to the second position, such as by pulling on one end of the suture. The second button then seats against the second distal side 320A of the second metacarpal 320.

With button 200 seated against second metacarpal 320, suture 110 is attached to the first button (120 or 200), which is then tightened to seat against the first proximal side 310B of first metacarpal 310.

The first button may also have been attached to suture 110 before pushing second button 200 through the opening, in which case the first button may be tightened to seat against the first proximal side 310B of first metacarpal 310 by loosening the suture 110, repositioning the first button, and then tightening suture 110 to the first button.

The surgeon then may optionally adjust the space between metacarpal bone 310 and metacarpal bone 320, prior to seating the first button against the first metacarpal.

Two or more devices 100 or 150 may be used for the support of a body part by another body part.

Some specific examples of the invention are as follows:

1. A method of carpometacarpal repair, the method comprising the steps of:

(a) forming an opening through a first metacarpal, the first metacarpal having a first proximal side and a first distal side, and a second metacarpal, the second metacarpal having a second proximal side and a second distal side;

(b) providing a device comprising a first button, a second button having a first position and second position, and a suture connected to at least the second button;

(c) passing the suture and second button through the opening so that the second button moves past the second distal side, where the second button moves to its second position, so the second button seats against the second distal side; and

(d) seating the first button against the first proximal side.

2. The method of example 1, wherein the first metacarpal is the thumb metacarpal.
3. The method of examples 1 or 2, wherein the second metacarpal is the index finger metacarpal.
4. The method of examples 1, 2 or 3, wherein the opening has a diameter of about 2.7 mm.
5. The method of example 1, wherein the opening has a diameter of about 1.0 mm to about 3.5 mm.
6. The method of any of examples 1-5, wherein the second button is biased towards the second position and automatically moves to the second position when moved past the second distal side.
7. The method of any of examples 1-5, wherein a surgeon moves the second button to the second position.
8. The method of example 7, wherein the second button is moved to the second position by the surgeon pulling on the suture.
9. The method of any of examples 1-8, wherein the opening is formed by pushing a K-wire through the first metacarpal and the second metacarpal.
10. The method of any of examples 1-8, wherein the opening is formed by pushing a K-wire through the first metacarpal and second metacarpal, and placing a cannulated drill over the K-wire and drilling a hole following the K-wire.
11. The method of example 10, further comprising the step of removing the K-wire from the cannulated drill bit prior to passing the suture and second button through the opening.
12. The method of any of examples 1-11, wherein the suture and second button are passed through the opening by pushing them with a K-wire.
13. The method of example 11, wherein the cannulated drill bit remains in the opening and the suture and second button are passed through them.
14. The method of any of examples 1-13 further comprising an initial step of removing or partially excising the trapezium.
15. The method of any of examples 1-14, wherein the carpometacarpal repair is at least one of carpometacarpal basal joint arthrosis, carpometacarpal arthritis, thumb instability and carpometacarpal fusion.
16. The method of any of examples 1-15, wherein the suture is formed as a loop.
17. The method of any of examples 1-16, wherein the first button has at least one hole to which the suture attaches.
18. The method of any of examples 1-16, wherein the first button has a plurality of holes to which the suture attaches.
19. The method of any of examples 1-18, wherein the second button has at least one hole to which the suture attaches.
20. The method of any of examples 1-18, wherein the second button has a plurality of holes to which the suture attaches.
21. The method of any of examples 1-20, wherein the first button is attached to the suture prior to passing the suture and second button through the opening.
22. The method of any of examples 1-21 that further comprises the step of adjusting the distance between the first metacarpal bone and the second metacarpal bone after the second button moves to the second position.
23. The method of example 21 that further comprises the steps of unattaching the suture from the first button and reattaching the suture to the first button in order to seat the first button to the first proximal side.
24. A device for supporting a body part by another body part, the device comprising:

(a) a first button;

(b) a suture having a first and second end;

(c) a second button attached to the second end of the suture, the second button having a first position and a second position.

25. The device of example 24, wherein the first button and second button each comprises one or more of plastic and metal.
26. The device of example 24, 25 or 26, wherein each button has a length between ⅛″ ½″.
27. The device of example 24, 25 or 26, wherein each button has a width between 1/64″ and ⅛″.
28. The device of example 24, 25 or 26, wherein each button has a width between ¼″ and ⅛″.
29. The method of any of examples 24-28, wherein each button has a thickness between 1/64″ and ⅛″.
30. The method of any of examples 24-28, wherein each button has a thickness between 1/64″ and ⅛″.
31. The method of any of examples 24-30, wherein there is a distance of 1½″ to 5″ between the first button and the second button when the buttons are each connected to the suture.
32. The method of any of examples 24-31, wherein the second button is biased towards the second position.
33. The device of any of examples 24-32, wherein the first button is connected to the first end of the suture.
34. The device of any of examples 24-32, wherein each button has two spaced-apart holes, and the suture is threaded through each of the spaced apart holes.
35. The device of any of examples 24-35, wherein the suture is formed of a material that is flexible and stretches when a force is applied to it.
36. The device of any of examples 24-35, wherein the suture material stretches when a force of at least ⅛ lb. is applied to it.
37. The device of any of examples 24-35, wherein the suture material stretches when a force of at least ¼ lb. is applied to it.
38. The device of any of examples 24-35, wherein the suture material stretches when a force of at least 1/16 lb. is applied to it.
39. The device of any of examples 24-38, wherein the suture material will support a load of at least 10 lbs.
40. The device of any of examples 24-38, wherein the suture material will support a load of at least 30 lbs.
41. The device of any of examples 24-40, wherein the second button has two openings and the suture passes through both of the openings.
42. The device of example 41, wherein the suture is flexible and stretches when a force of ⅛ lb. or greater is applied to it.
43. The device of any of examples 24-42, wherein the suture is formed in a loop with two ends at the first button.
44. The device of any of examples 24-42, wherein the suture can support a load of at least 10 lbs.
45. A method of carpometacarpal repair, the method comprising the steps of:

(e) forming an opening through a first body part, the first body part having a first proximal side and a first distal side, and a second body part, the second body part having a second proximal side and a second distal side;

(f) providing a device comprising a first button, a second button having a first position and second position, and a suture connected to at least the second button;

(g) passing the suture and second button through the opening so that the second button moves past the second distal side, where the second button moves to its second position, so the second button seats against the second distal side; and

(h) seating the first button against the first proximal side.

46. The method of example 1, wherein the first body part is the thumb body part.
47. The method of examples 1 or 2, wherein the second body part is the index finger body part.
48. The method of examples 1, 2 or 3, wherein the opening has a diameter of about 2.7 mm.
49. The method of example 1, wherein the opening has a diameter of about 1.0 mm to about 3.5 mm.
50. The method of any of examples 1-5, wherein the second button is biased towards the second position and automatically moves to the second position when moved past the second distal side.
51. The method of any of examples 1-5, wherein a surgeon moves the second button to the second position.
52. The method of example 7, wherein the second button is moved to the second position by the surgeon pulling on the suture.
53. The method of any of examples 1-8, wherein the opening is formed by pushing a K-wire through the first body part and the second body part.
54. The method of any of examples 1-8, wherein the opening is formed by pushing a K-wire through the first body part and second body part, and placing a cannulated drill over the K-wire and drilling a hole following the K-wire.
55. The method of example 10, further comprising the step of removing the K-wire from the cannulated drill bit prior to passing the suture and second button through the opening.
56. The method of any of examples 1-11, wherein the suture and second button are passed through the opening by pushing them with a K-wire.
57. The method of example 11, wherein the cannulated drill bit remains in the opening and the suture and second button are passed through them.
58. The method of any of examples 1-13 further comprising an initial step of removing or partially excising the trapezium.
59. The method of any of examples 1-14, wherein the carpometacarpal repair is at least one of carpometacarpal basal joint arthrosis, carpometacarpal arthritis, thumb instability and carpometacarpal fusion.
60. The method of any of examples 1-15, wherein the suture is formed as a loop.
61. The method of any of examples 1-16, wherein the first button has at least one hole to which the suture attaches.
62. The method of any of examples 1-16, wherein the first button has a plurality of holes to which the suture attaches.
63. The method of any of examples 1-18, wherein the second button has at least one hole to which the suture attaches.
64. The method of any of examples 1-18, wherein the second button has a plurality of holes to which the suture attaches.
65. The method of any of examples 1-20, wherein the first button is attached to the suture prior to passing the suture and second button through the opening.
66. The method of any of examples 1-21 that further comprises the step of adjusting the distance between the first body part bone and the second body part bone after the second button moves to the second position.
67. The method of example 11, wherein the cannulated drill bit includes a longitudinal slit, and that further includes the step of removing the cannulated drill bit from the opening and removing the suture from the cannulated drill bit through the longitudinal slit.
68. The method of example 11 that further includes the step of removing the cannulated drill bit from the opening.
69. The method of example 54, wherein the cannulated drill bit includes a longitudinal slit, and that further includes the step of removing the cannulated drill bit from the opening and removing the suture from the cannulated drill bit through the longitudinal slit.
70. The method of example 54 that further includes the step of removing the cannulated drill bit from the opening.

Having thus described some embodiments of the invention, other variations and embodiments that do not depart from the spirit of the invention will become apparent to those skilled in the art. The scope of the present invention is thus not limited to any particular embodiment, but is instead set forth in the appended claims and the legal equivalents thereof. Unless expressly stated in the written description or claims, the steps of any method recited in the claims may be performed in any order capable of yielding the desired result.

What is claimed is:

1. A method of carpometacarpal repair, the method comprising the steps of:
(i) forming an opening through a first metacarpal, the first metacarpal having a first proximal side and a first distal side, and a second metacarpal, the second metacarpal having a second proximal side and a second distal side;
(j) providing a device comprising a first button, a second button having a first position and second position, and a suture connected to at least the second button;
(k) passing the suture and second button through the opening so that the second button moves past the second distal side, where the second button moves to its second position, so the second button seats against the second distal side; and
(l) seating the first button against the first proximal side.

2. The method of claim 1, wherein the first metacarpal is the thumb metacarpal.

3. The method of claim 1, wherein the second metacarpal is the index finger metacarpal.

4. The method of claim 1, wherein the opening has a diameter of about 2.7 mm.

5. The method of claim 4, wherein the opening has a diameter of about 1.0 mm to about 3.5 mm.

6. The method of claim 1, wherein the second button is biased towards the second position and automatically moves to the second position when moved past the second distal side.

7. The method of claim 1, wherein a surgeon moves the second button to the second position.

8. The method of claim 7, wherein the second button is moved to the second position by the surgeon pulling on the suture.

9. The method of claim 1, wherein the opening is formed by pushing a K-wire through the first metacarpal and the second metacarpal.

10. The method of claim 1, wherein the opening is formed by pushing a K-wire through the first metacarpal and second metacarpal, and placing a cannulated drill over the K-wire and drilling a hole following the K-wire.

11. The method of claim 10, further comprising the step of removing the K-wire from the cannulated drill bit prior to passing the suture and second button through the opening.

12. The method of claim 1, wherein the suture and second button are passed through the opening by pushing them with a K-wire.

13. The method of claim 11, wherein the cannulated drill bit remains in the opening and the suture and second button are passed through it.

14. The method of claim 1 further comprising an initial step of removing or partially excising the trapezium.

15. The method of claim 1, wherein the carpometacarpal repair is at least one of carpometacarpal basal joint arthrosis, carpometacarpal arthritis, thumb instability and carpometacarpal fusion.

16. The method of claim 1, wherein the suture is a formed as a loop.

17. The method of claim 1, wherein the first button has at least one hole to which the suture attaches.

18. The method of claim 1, wherein the first button has a plurality of holes to which the suture attaches.

19. The method of claim 15, wherein the second button has at least one hole to which the suture attaches.

20. The method of claim 16, wherein the second button has a plurality of holes to which the suture attaches.

21. The method of claim 1, wherein the first button is attached to the suture prior to passing the suture and second button through the opening.

22. The method of claim 1 that further comprises the step of adjusting the distance between the first metacarpal bone and the second metacarpal bone after the second button moves to the second position.

23. The method of claim 21 that further comprises the steps of unattaching the suture from the first button and reattaching the suture to the first button in order to seat the first button to the first proximal side.

24. A device for supporting a body part by another body part, the device comprising:
(d) a first button;
(e) a suture having a first and second end;
(f) a second button attached to the second end of the suture, the second button having a first position and a second position.

25. The device of claim 24, wherein the first button and second button comprises one or more of plastic and metal.

26. The device of claim 24, wherein each button has a length between ⅛″ ½″.

27. The device of claim 24, wherein each button has a width between 1/64″ and ⅛″.

28. The device of claim 24, wherein each button has a width between ¼″ and ⅛″.

29. The device of claim 24, wherein each button has a thickness between 1/64″ and ⅛″.

30. The device of claim 26, wherein each button has a thickness between 1/64″ and ⅛″.

31. The device of claim 24, wherein there is a distance of 1½″ to 5″ between the first button and the second button when the buttons are each connected to the suture.

32. The device of claim 24, wherein the second button is biased towards the second position.

33. The device of claim 24, wherein the first button is connected to the first end of the suture.

34. The device of claim 24, wherein each button has two spaced-apart holes, and the suture is threaded through each of the spaced apart holes.

35. The device of claim 24, wherein the suture is formed of a material that is flexible and stretches when a force is applied to it.

36. The device of claim 24, wherein the suture material stretches when a force of at least ⅛ lb. is applied to it.

37. The device of claim 24, wherein the suture material stretches when a force of at least ¼ lb. is applied to it.

38. The device of claim 24, wherein the suture material stretches when a force of at least 1/16 lb. is applied to it.

39. The device of claim 24, wherein the suture material will support a load of at least 10 lbs.

40. The device of claim 24, wherein the suture material will support a load of at least 30 lbs.

41. The device of claim 24, wherein the second button has two openings and the suture passes through both of the openings.

42. The device of claim 24, wherein the suture is flexible and stretches when a force of ⅛ lb. or greater is applied to it.

43. The device of claim 24, wherein the suture is formed in a loop with two ends at the first button.

44. The device of claim 24, wherein the suture can support a load of at least 10 lbs.

45. The method of claim 11, wherein the cannulated drill bit includes a longitudinal slit, and that further includes the step of removing the cannulated drill bit from the opening and removing the suture from the cannulated drill bit through the longitudinal slit.

46. The method of claim 11 that further includes the step of removing the cannulated drill bit from the opening.

47. Tie first button

48. Second button biased

49. Define first position and second position

50. Force supported by suture

51. Shape of buttons

52. Buttons can be different

53. Type of drill bit

54. Define width, length and thickness
